(19) United States
(12) Patent Application Publication
(10) Pub. No.: US 2016/0120835 A1
(43) Pub. Date: May 5, 2016

(54) A TOPICAL COMPOSITION

(71) Applicant: LEO LABORATORIES LIMITED, Dublin 12 (IE)

(72) Inventors: Edit FARKAS, Herlev (DK); Chinar Sami SAEED, Nærum (DK); Mikkel KROGH-MADSEN, Ballerup (DK); Per-Ola ARVIDSSON, Ballerup (DK)

(73) Assignee: LEO LABORATORIES LIMITED, Dublin 12 (IE)

(21) Appl. No.: 14/897,270

(22) PCT Filed: Jun. 12, 2014
(86) PCT No.: PCT/EP2014/062188

§ 371 Date: Dec. 10, 2015

(30) Foreign Application Priority Data
Jun. 12, 2013 (EP) 13171724.1

(51) Int. Cl. A61K 31/22; A61K 9/00; A61K 9/107

(52) U.S. Cl. CPC A61K 31/22; A61K 9/107; A61K 9/0014

(57) ABSTRACT

The invention provides a topical composition for cutaneous application which is an oil-in-water emulsion comprising (a) an oily phase, (b) an ingenol derivative in dissolved form; (c) an aqueous phase buffered to a p H of below 4.5, (d) at least one surfactant.

### FIELD OF INVENTION

The present invention relates to a topical pharmaceutical formulation comprising a pharmacologically active agent, one or more surfactants, an aqueous phase and an oily phase.

### BACKGROUND OF THE INVENTION

The invention provides a pharmaceutical formulation suitable for topical application of the compound ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester; PEP005). Ingenol-3-angelate (PEP005) is a protein kinase C activator in phase III clinical development for the treatment of actinic keratosis. The drug candidate is also in phase II trials for non-melanoma skin cancer [Ogbourne, S. M.; Anti-cancer Drugs, (2007), 18, 357-62].

The compound ingenol-3-angelate (PEP005) [Sayed, M. D. et. al.; Experienta, (1980), 36, 1206-1207] can be isolated from various Euphorbia species, and particularly from Euphorbia peplus [Hohmann, J. et. al; Planta Med., (2000), 66, 291-294] and Euphorbia drummondii by extraction followed by chromatography as described in U.S. Pat. No. 7,449,492.

Pharmaceutical formulation of the compound has been described in WO200768963.

Angelic acid and angelic acid esters such as ingenol-3-angelate, are prone to isomerisation of the double bond to form the tiglate ester, particularly at basic pH or when subjected to heat [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563]. As a consequence only carefully optimised conditions for ester formation can be applied in the synthetic preparation of ingenol-3-angelate.

Furthermore, ingenol-3-acylates are known to be unstable as they rearrange to afford the ingenol-5-acylates and ingenol-20-acylates [Sorg, B. et. al, Z. Naturforsch., (1982), 37B, 748-756].

WO 2007/068963 discloses a gel formulation for the treatment of skin cancer in which ingenol angelate is dissolved in an aprotic solvent, the formulation further comprising an acidifying agent such that the pH of the formulation is no greater than 4.5. The aqueous gel is generally stored at refrigeration temperature.

One object of the invention is therefore to provide a composition of the ingenol derivative which is stable at room temperature for the entire shelf-life of the composition.

Another object of the invention is to provide a composition exhibiting favourable penetration characteristics and biological activity.

A further object of the invention is to provide a composition with reduced skin irritation and favourable cosmetic properties and improved patient compliance.

### SUMMARY OF THE INVENTION

In the research leading to the present invention, it was an object to identify oil-in-water formulation. With water being the continuous phase, the patients often prefer a oil-in water formulation due to the non-greasy and non-sticky sensation of a water based formulation, compared to an oil-based formulation.

In one aspect, the present invention relates to a topical composition for cutaneous application which is an oil-in-water emulsion comprising:

(a) an oily phase,
(b) an ingenol derivative in dissolved form;
(c) an aqueous phase buffered to a pH of below 4.5,
(d) at least one surfactant.

Ingenol derivatives such as ingenol-3-angelate are known to be extremely sensitive to higher pH conditions (pH above about 4.5 in aqueous compositions or alkaline reacting substances in non-aqueous compositions) which contribute to the isomerization of the angelic acid ester to the tiglate ester and the acyl migration of the angelic acid moiety. To ensure an adequate chemical stability of the substance throughout the shelf-life of the composition, it should include an acidic compound capable of neutralizing alkaline impurities which may be present in one or more of the excipients of the composition and which are detrimental to the chemical stability of the ingenol derivative.

The present composition has been found to result in improved chemical stability of the ingenol derivative included therein permitting the composition to be stored at room temperature (about 25° C.) throughout its shelf-life. The improved stability may be the result of partitioning of the ingenol derivative to the lipid/oily phase of the oil-in-water emulsion due to its extremely low water solubility, thus protecting it from chemical interaction with reactive components in the aqueous phase.

Human skin, in particular the outer layer, the stratum corneum, provides an effective barrier against penetration of microbial pathogens and toxic chemicals. While this property of skin is generally beneficial, it complicates the dermal administration of pharmaceuticals in that a large quantity, if not most, of the active ingredient applied on the skin of a patient suffering from a dermal disease may not penetrate into the viable layers of the skin (the dermis and epidermis) where it exerts its activity. The present composition has been found to exhibit improved penetration of the ingenol derivative into the viable layers of the skin, but not higher permeation through the skin than seen with the hydrogel formulation disclosed in WO 2007/068963 despite containing a lower amount of an alcohol such as isopropanol as a solvent or no alcohol at all.

In another aspect of the invention, the present composition may be used in the treatment of a dermal disease or condition.

### DETAILED DESCRIPTION OF THE DRAWINGS

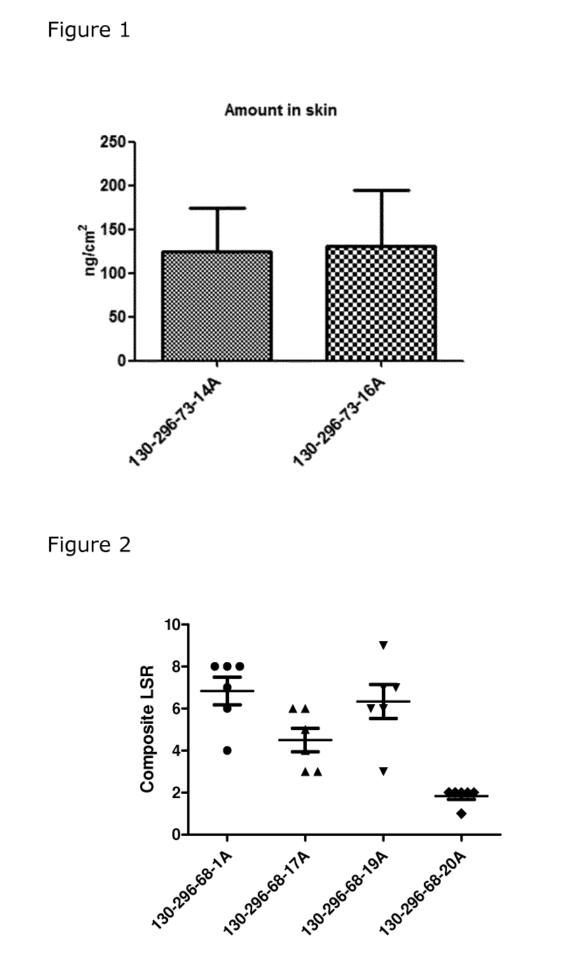

FIG. 1 shows the results of the permeation and penetrations study of example 3.

FIG. 2 shows the results of the local skin reaction study of example 4.

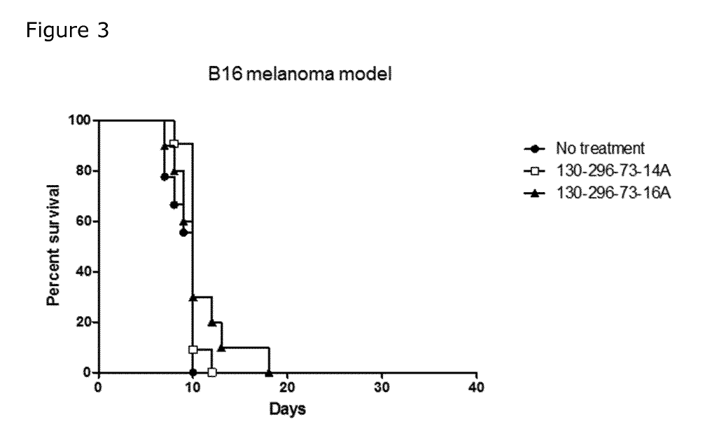

FIG. 3 shows the results of the B16 model of example 5.

### DETAILED DESCRIPTION OF THE INVENTION

### Definitions

In the present context, the term “oil-in-water emulsion” is intended to include a formulation containing an oily phase and an aqueous phase, wherein the oil phase is dispersed in the water continuous phase. The ingenol derivative is present mostly in the oily phase and in the interphase with the aqueous phase.

The term “surfactant” is intended to indicate a surfactant compatible with an oil-in-water composition, such as having a surface activity. Mixtures of surfactants with an HLB value of 9-18 and 2-12 can be applied for preparation of oil-in-water systems. Additionally, the use of polymers with surface activity can also create oil-in-water systems.

The term “ingenol derivative” is intended to mean an ingenol compound isolated from a species of Euphorbia, in particular from E. peplus, or an ingenol derivative prepared by chemical synthesis or by a semi-synthetic route, e.g. as disclosed in pending application No. PCT/DK2011/000081. Examples of ingenol derivatives that may be included in the present compositions are ingenol-3-angelate, ingenol-5-angelate, ingenol-20-angelate, 20-O-acetyl-ingenol-3-angelate and 20-deoxy-ingenol-3-angelate. Ingenol-3-angelate, also known as ingenol-3-mebutate or PEP 005, is currently on the market for the treatment of actinic keratosis.

The term “storage stability” is intended to indicate that the composition exhibits chemical and physical stability characteristics that permit storage of the composition, at refrigeration or, preferably, room temperature for a sufficient period of time (the shelf-life of the composition) to make the composition commercially viable, such as at least 12 months, in particular at least 18 months, and preferably at least 2 years.

The term “chemical stability” or “chemically stable” is intended to indicate that no more than 10%, preferably no more than 6%, of the ingenol derivative degrades over the shelf-life of the product, typically 2 years. An approximation of chemical stability

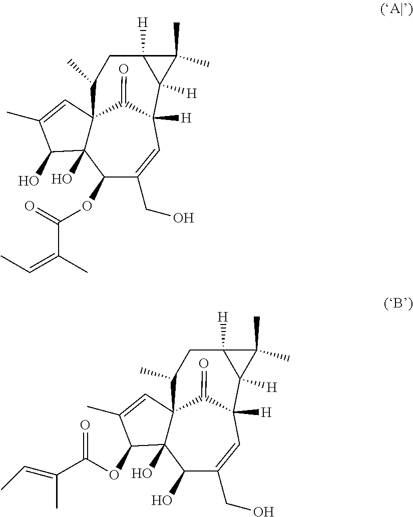

at room temperature is obtained by subjecting the composition to accelerated stability studies at 40° C. for 3 months or by subjecting the composition to stability studies at 25° C. for 6 months. If less than about 2.5% of the substance, e.g. ingenol-3-angelate, has degraded after 3 months at 40° C., a shelf-life of 2 years at room temperature is considered to be feasible. If less than about 2.5% of the substance, e.g. ingenol-3-angelate, has degraded after 6 months at 25° C. then a shelf-life of 2 years at room temperature is expected, i.e. less than 10% of the ingenol-3-angelate will be expected to degrade over a storage period of 2 years at 25° C. These studies are carried out according to ICH humidity guideline, at conditions of 25° C.±2°/60% RH±5% and/or 40° C.±2°/75% RH±5%, in hermetically sealed containers. Preferred chemically stable gels include after storage for 2 years at 25° C., less than 5% by weight of total ingenanes in the composition are ‘A’ and/or ‘B’. Thus, if the total amount of ‘A’ and ‘B’ exceeds 5% by weight of the total ingenanes, the gel's shelf-life is not ideal.

The term “physical stability” or “physically stable” is intended to mean that the composition retains its macroscopic and microscopic appearance over the shelf-life of the product, e.g. that the ingenol derivative does not precipitate from the solvent phase or that there is no visible phase separation of the solvent phase and the carrier phase.

The term “solubilization capacity” is intended to indicate the ability of a solvent or mixture of solvents to dissolve a given substance, expressed as the amount required to effect complete solubilization of the substance.

The term “skin penetration” is intended to mean the diffusion of the active ingredient into the different layers of the skin, i.e. the stratum corneum, epidermis and dermis.

The term “skin permeation” is intended to mean the flux of the active ingredient through the skin into the systemic circulation or, in case of in vitro studies, the receptor fluid of the Franz cell apparatus used in the experiment.

The term “medium chain triglycerides” is intended to indicate triglyceride esters of fatty acids with a chain length of 6-12 carbon atoms. A currently favoured example of medium chain triglycerides is a mixture of caprylic (C₈) and capric (C₁₀) triglycerides. e.g. available under the trade name Miglyol 812.

The term “acidic compound” is intended to indicate a compound capable of providing a net overall acidic environment in the composition and/or capable of neutralizing alkaline impurities detrimental to the stability of the ingenol derivative.

The term “fatty alcohol”, “fatty alcohol ether”, “fatty acid ester” and “fatty acid” refers to a functional group attached to an aliphatic chain. The chain can be saturated or unsaturated, and may contain multiple double bonds. In embodiments of the invention the aliphatic chain is a C₆₋₂₂ chain. In embodiments of the invention the aliphatic chain containing even numbers of carbon.

The term “lower alcohol” in the context of the present invention refers to a C₂₋₂₂ alcohol, wherein the carbon chain may be straight or branched and optionally containing double bonds.

### EMBODIMENTS

In an embodiment of the invention, the oily phase contains at least one solvent.

The pH of the aqueous phase will typically be below 4.5 or between about 2 to about 4.5, e.g. pH 2, 2.5, 3, 3.5, 4, or 4.5. In an embodiment the buffer having a pH of from about 2 to about 3 is used, because this pH range may permit the composition to be stored at room temperature (25° C.) for extended periods. For instance, in embodiments the pH of the buffer is 2.0, 2.1, 2.2, 2.3, 2.4, 2.5, 2.6, 2.7, 2.8, 2.9 or 3.0. In an embodiment the aqueous phase is buffered to a pH of 2.6-3.7.

In an embodiment the invention provides the composition as above, wherein the surfactant is selected from the following surfactant groups of PEG sorbitane fatty acid esters, PEG fatty alcohol ethers, PEG fatty acid esters, PEG castor oil derivatives, PEG glicerides, propylene glycol esters, sucrose esters, poloxamers and phospholipids. In an embodiment the surfactants are polyethylene glycol C₆₋₂₀ fatty acid glyceride selected from the group consisting of caprylocaproyl PEG glyceride, lauroyl PEG glyceride, linoeoyl PEG glyceride, oleoyl PEG glyceride and stearoyl PEG glyceridepolyoxyethylene C₈₋₂₀ alkyl ether selected from the group consisting of PEG monocetyl ether, PEG monolauryl ether, PEG monooleyl ether and PEG monostearyl ether (such as polyoxyethylene-2-stearyl ether), a polysorbate selected from the group consisting of polysorbate 20, 40, 60 and 80, or a polyoxyethylene castor oil derivative such as polyoxyl castor oil or hydrogenated polyoxyl castor oil.

In an embodiment the invention provides the composition according to any one of the above embodiments, wherein the surfactant is selected from PEG 15 hydroxy stearate, poloxamers such as Lutrol F127, Lutrol F68, phospholipid such as Lipoid S100, Vitamine E PEG succinate, and a mixture of acrylamide acryloyldimethyl taurate copolymer, isohexadecane and polysorbate 80 (Sepineo P600).

To maintain good physical stability of the composition, in particular to avoid separation of the aqueous and lipid phases therein, it may be advantageous to include another surfactant. In an embodiment the invention provides the composition according to any of the embodiments above, wherein the composition contains additional surfactants, wherein the additional surfactant is selected from fatty alcohol, sucrose esters, mono-di-tri-glyceride or polyglycerol derivatives.

In an embodiment the invention provides the composition according to the embodiment above wherein the surfactant is selected from cetostearyl alcohol, sucrose stearate and palmitate esters, caprylic/capric glycerides, glyceryl monooleate, and triglycerol diisostearate.

In an embodiment the invention provides the composition of any of the embodiments above, wherein the surfactant is present in an amount of from about 0.1% by weight to about 20% by weight of the composition.

In an embodiment the invention provides the composition according to the embodiment above, wherein the surfactant or surfactants are present in a total concentration of from about 0.5% by weight to about 8% by weight, or from about 1% by weight to about 7% by weight, such as about 2.5% by weight, of the composition.

In an embodiment the invention provides the composition according to any of the embodiments above, wherein the ingenol derivative is selected from the group consisting of ingenol-3-angelate, ingenol-5-angelate, ingenol-20-angelate, 20-O-acetyl-ingenol-3-angelate and 20-deoxy-ingenol-3-angelate.

In an embodiment the invention provides the composition according to the embodiment above, wherein the ingenol derivative is ingenol-3-angelate.

In an embodiment the invention provides the composition according to any of the embodiments above, wherein the oily phase of the composition contains at least one solvent.

In an embodiment the invention provides the composition according to the embodiment above, wherein the oily solvent is selected from vegetable oil, e.g. sesame oil, sunflower oil, palm kernel oil, corn oil, safflower oil, olive oil, avocado oil, jojoba oil, grape kernel oil, almond oil, canola oil, coconut oil, cottonseed oil, peanut oil, walnut oil, soybean oil or wheat germ oil, a highly purified vegetable oil, e.g. medium chain triglycerides, long chain triglycerides, castor oil, caprylic/capric mono- and diglycerides or caprylic/capric mono-, di- and triglycerides, a synthetic oil, e.g. isopropyl myristate, isopropyl palmitate, isopropyl linoleate, isopropyl monooleate, isostearyl isostearate or polyoxypropylene stearyl ether (such as polyoxypropylene-15-stearyl ether), a propylene glycol derivative such as propylene glycol dicaprylate dicaprate (such as Labrafac, Capryol 90 and Crodamol PC), and alkyl or dialkyl ester such as ethyl oleate, diisopropyl adipate, dicaprylyl carbonate or propylene carbonate, or a C₁₀₋₃₀ cholesterol or lanosterol ester, Silicone oils such as cyclomethicone 5-NF and dimethicone.

I an embodiment the compositions contain a co-solvent which is selected from the group consisting of alcohols such as benzyl alcohol, as ethanol, n-propanol, isopropanol, n-butanol, 2-butanol or benzyl alcohol, or diols such as propylene glycol, or fatty alcohols such as oleoyl alcohol, stearyl alcohol and cetyl alcohol.

In an embodiment the invention provides the composition according to the embodiments above wherein the oily solvent is present in a concentration of about 0.5-80%, in particular 5-50%.

In an embodiment the invention provides the composition according to any one of the embodiments above, further comprising a penetration enhancer such as a lower alcohols or diols, pyrrolidones, essential oils, azones, isopropyl esters, propylene glycol esters and surfactants.

In the present invention, a combination of surfactants can be present. In that case the range above optionally applies to each surfactant.

Some surfactants are available as combination products. This applies to for example Sepineo SE68, which is a mixture of cetearyl alcohol and cetearyl glucosid, Polawax NF, which is a mixture of cetearyl alcohol and polysorbate 60, and Crodafos CES, which is a mixture of cetearyl alcohol, dicetyl phosphate and ceteth-10 phosphate.

For the purpose of this invention the range above applies also to the combined surfactants as well as for single surfactant systems.

It is generally preferred to include the lower alcohol or diol solvent is low amounts in order to enhance penetration into the skin. Thus the lower alcohol or diol solvent may be present in an amount of 0.1-20% by weight, preferably 0.5-10% by weight of the composition.

As the lower alcohols may cause irritation of the skin and therefore compounds such as glycerol, hyaluronic acid and sorbitol for example may be added to reduce this.

In an embodiment, the composition further comprising an occlusive agent selected from the group consisting of a mineral oil, e.g. liquid paraffin, or at least one paraffin selected from paraffins consisting of hydrocarbons with chain lengths from C₅ to C₆₀, the chain lengths peaking at C₁₄₋₁₆, C₁₈₋₂₂, C₂₀₋₂₂, C₂₀₋₂₆, C₂₈₋₄₀, and C₄₀₋₄₄, or mixtures thereof or an iso-paraffin such as isohexadecane or squalane, or petrolatum-polyethylene blend.

To impart a desired viscosity to the present composition, it may suitably include a viscosity-increasing ingredient.

The amount of viscosity-increasing ingredient may vary according to the viscosifying power of the ingredient, but may typically be in the range of about 0.1-10% by weight of the composition.

If the surfactant included in the composition is from the polyacrylate group or from polyacrylamides such as SEPINEO P600, it may in itself impart a suitable viscosity. SEPINEO P600 may be included in an amount of about 1-10% by weight, such as about 2.5% by weight, of the composition. Also viscosity modifiers such as polysaccharides such as hyaluronic acid, chitosan, pectin, xanthan gum, agar, carrageenan, tragacanth, starch, polydextrose cellulose derivative such as HEC, HPC, HPMC, MC, CMC-Na, polyacrylates, polycarbophyl, polyvinylalcohol, polyvinylpirrolydones can be applied in a preferred embodiment.

Other viscosity enhancers such as PEG derivative or wax may also be used. The PEG derivative maybe PEG 100 distearate, the wax may be a mineral wax composed of a mixture of high molecular weight hydrocarbons, e.g. saturated C₃₅₋₇₀ alkanes, such as microcrystalline wax. Alternatively, the wax may be a vegetable or animal wax, e.g. esters of C₁₄₋₃₂ fatty acids and C₁₄₋₃₂ fatty alcohols, such as beeswax or hydrogenated castor oil. Alternatively, the viscosity-increasing ingredient may be an inorganic substance such as fumed silica, e.g. available under the trade name Aerosil.

The acidic compound included in the present composition may favourably be selected from a buffer such as a citrate or acetate buffer which may be included in an amount of about 0.1-2% by weight of the composition.

In an alternative embodiment another water-soluble acidic compound such as a hydroxy acid, e.g. lactic acid, salicylic acid or glycolic acid. Neutralization of alkaline reacting substances may also be provided by, e.g., fumed silica, which may be included in the composition in an amount of about 1-13% by weight such as about 3-9% by weight. Alternatively, neutralization of alkaline reacting substances may be provided by addition of a fatty acid such as oleic acid, linoleic acid, stearic acid, lauric acid, palmitic acid, capric acid, caprylic acid, pelargonic acid or enanthic acid to the composition.

The amount of water in the composition may range from about 10% to about 95% by weight, e.g. from about 20% to about 90% by weight of the composition.

Examples of ingenol derivatives that may be included in the present composition are ingenol-3-angelate, ingenol-5-angelate, ingenol-20-angelate, 20-O-acetyl-ingenol-3-angelate and 20-deoxy-ingenol-3-angelate. A currently favoured ingenol derivative is ingenol-3-angelate, also known as ingenol-3-mebutate or PEP 005. The ingenol derivative may be included in the composition in an amount of about 0.001-0.5% by weight of the composition.

The composition of the invention may be used in the topical treatment of a dermal disease or condition. Examples of dermal diseases and conditions are actinic keratosis, seborrheic keratosis, skin cancer, such as basal cell carcinoma or squamous cell carcinoma, warts, keloids, scars, photoaged or photodamaged skin, or acne.

The term “skin cancer” is intended to include non-melanoma skin cancer, malignant melanoma, Merkel cell carcinoma, squamous cell carcinoma or basal cell carcinoma. Basal cell carcinomas include superficial basal cell carcinoma as well as nodular basal cell carcinoma.

The term “photodamaged skin” is intended to include cover fine lines, wrinkles and UV-ageing. UV ageing is often manifested by an increase in the epidermal thickness or epidermal atrophy and most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting in a leathery appearance, skin fragility and irregular pigmentation, loss of tone and elasticity, as well as wrinkling, dryness, sunspots and deep furrow formation.

The term “warts” in the context of the present invention is intended to human papilloma virus (HPV) infections leading to formation of warts on the body, such as the skin, genitals and mouth.

The present composition may also be effective at reducing or minimizing scar tissue or improving cosmesis or functional outcome in a wound and scar reduction, wherein the wound is cutaneous, chronic or for example diabetes associated, and includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant donor and recipient sites, immune response conditions, oral wounds, stomach or intestinal wounds, damaged cartilage or bone, amputation sides and corneal lesions.

The potency of a composition of the invention may be tested in a model where test compositions (20 μL) are applied topically, once daily, on a 2 cm² area on each flank of anaesthetized CRL:CD(SD)-HR-CD male rats (12 weeks old). 6 different animals are treated with each formulation. Animals are allowed to recover from anaesthesia after 2 hours. Dosing with formulations may vary from a single application to several, once daily applications. A visual scoring on erythema, oedema, ulceration and telangiectasia is performed 24 hrs after each application. 24 hours after the last application, animals are euthanized by asphyxiation in CO₂ and two 5 mm punch biopsies are taken from each treatment site: one biopsy is snap frozen in liquid nitrogen and stored at −20° C. until analysis for the chemokine KC (CXCL1), the other sample is fixated in formalin at room temperature for histological analysis. Clinical scoring, KC production and histological evaluation of the epidermal and dermal compartment are compared against Picato gel formulation in order to identify new formulations with the same ability to create a strong, local skin reaction, increase KC production and induce necrosis of epidermis and dermis. An increase in any of these parameters is interpreted as an increased potency of the formulation.

The invention is described in further detail in the following examples which are not in any way intended to limit the scope of the invention as claimed.

### Examples 1A

Amount mg/g

| Components | 68-01A | 68-17A | 68-19A | 68-20A |
|---|---|---|---|---|
| Ingenol | 0.15 | 0.15 | 0.15 | 0.15 |
| mebutate | | | | |
| Benzyl alcohol | 5 | 5 | 5 | 5 |
| Sepineo P 600 | 25 | 25 | 25 | 25 |
| Natrosol + | | | | |
| 330CS | | | | |
| Propylene | 210 | | | |
| glycol | | | | |
| Medium chain | 554.5 | 554.5 | | |
| triglycerides | | | | |
| Isopropyl | | | 210 | |
| myristate | | | | |
| Cyclomethicone | | | | 554.5 |
| 5-NF | | | | |
| Polysorbate 80 | 5 | 5 | 5 | 5 |
| Citrate buffer | 200.35 | 410.35 | 754.85 | 410.35 |

### Examples 1B

| | | | 08- | | | |
|---|---|---|---|---|---|---|
| | 08-07A | 08-08A | 09A | 08-10A | 08-14A | 08-16A |
| Components | mg/g | | | | | |
| Ingenol | 0.5 | 0.5 | 0.5 | 0.5 | 0.5 | 0.5 |
| mebutate | | | | | | |
| Benzyl Alcohol | 5 | 5 | 5 | 5 | 5 | 5 |
| Citrate buffer pH | | | | | | 400 |
| 2.3 | | | | | | |
| Citrate buffer pH | | 300 | | | 400 | |
| 2.7 | | | | | | |
| Citrate buffer pH | 400 | | 500 | 600 | | |
| 3.0 | | | | | | |
| Propylene glycol | 170 | 270 | 70 | 70 | 220 | 110 |
| Sepineo P600 | 25 | 25 | 25 | 25 | 25 | 35 |
| Medium chain | 399.5 | 399.5 | 399.5 | 299.5 | 299.5 | 149.5 |
| triglyceride | | | | | | |
| Isopropyl alcohol | | | | | | 300 |
| Oleic acid | | | | | 50 | |

### Study Protocol 08—Preparation Method

1. Drug phase: ingenol mebutate was solubilised in Benzyl Alcohol. (Drug phase solution can be used for 3 days if stored dark (UV protected) in glass vessel at 2-8° C.).
2. Buffer stock solution 1% (w/w): Citric acid and Na-Citrate were weighed and dissolved in the weighed amount of water. pH was adjusted with either Citric acid or NaOH solutions. (Buffer stock solution was stored at 2-8° C.).
3. Aqueous phase: Buffer stock and Propylene Glycol (and IPA, if existed) were mixed in an appropriate container
4. Oil phase: Miglyol 812 (and oleic acid—melting point 40° C., if existed) and Sepineo P600 were mixed. The mixture was homogenized
5. Final formulation: Drug phase was added to Oil phase under mixing. Then the Aqueous phase was slowly added to Oil phase under homogenisation.

### Examples 1C

| | |
|---|---|
| 15-17A | 15-18A |

| | |
|---|---|
| Components | mg/g |

| | | |
|---|---|---|
| Ingenol mebutate | 0.5 | 0.5 |
| Benzyl alcohol | 10 | 10 |
| Crodafos CES | 50 | |
| Emulsifying wax (Polawax | | 50 |
| NF) | | |
| Isohexadecane | 50 | 50 |
| Isopropyl myristate | 40 | 40 |
| Arlamol E | 30 | 30 |
| Glycerol 85% | 30 | 30 |
| Citrate buffer 1% | 789.5 | 789.5 |

1. Drug phase: ingenol mebutate was solubilised in Benzyl Alcohol.
2. Buffer stock solution 1% (w/w): Citric acid and Na-Citrate were weighed and dissolved in the weighed amount of water. pH was adjusted with either Citric acid or NaOH solutions.

| Stock solution | |
|---|---|
| Components | mg/g |
| Citric acid | 8 |
| Na-citrate dihydrate | 2 |
| NaOH | Adjust pH to 2.75 |
| Water purified | qs |

3. Aqueous phase: The amount of the buffer was weighed, and heated to 60° C.
4. Oil phase: The surfactant (Crodafos CES, Emulsifying wax, Arlamol HD, Isopropylmyristate, Arlamol E, and glycerol were melted together by heating to 60° C.
5. Emulsification: The oil phase and the Aqueous phase were blended by moderate mixing. The mixture was homogenized with Silverson L4RT until homogeneity. Moderate mixing was applied until the emulsion cooled down below 30° C.
6. Final formulation: Drug phase was added to Emulsion under mixing.

### Examples 1D

| | mg/g | | | | | | |
|---|---|---|---|---|---|---|---|
| | | 21- | 21- | 21- | | 21- | |
| Components | 21-01A | 02A | 05A | 12A | 21-13A | 16A | 21-17A |
| Ingenol | 0.5 | 0.5 | 0.5 | 0.5 | 0.5 | 0.5 | 0.5 |
| mebutate | | | | | | | |
| Benzyl Alcohol | 5 | 5 | 5 | 5 | 5 | 5 | 5 |
| Citrate buffer | 600 | 600 | 400 | 200 | 200 | 600 | 400 |
| (1%) pH 2.7 | | | | | | | |
| Sepineo P600 | 25 | 25 | 25 | 25 | 25 | 25 | 25 |
| Polysorbate | | 5 | 5 | 5 | 5 | 5 | 5 |
| 80 | | | | | | | |
| Propylene | 210 | 210 | 210 | 210 | 310 | 210 | 210 |
| glycol | | | | | | | |
| Medium chain | 159.5 | 104.5 | 354.5 | 559.5 | 459.5 | 104.5 | 154.5 |
| triglycerides | | | | | | | |
| Akoline MCM | | 50 | | | | | |
| Imwitor 742 | | | | | | 50 | |
| Isopropyl | | | | | | | 200 |
| myristate | | | | | | | |
| Macrogol 2 | | | | | | | |
| stearyl ether | | | | | | | |

| | mg/g | | | | |
|---|---|---|---|---|---|
| Components | 21-18A | 21-21A | 21-22A | 21-23A | 21-24A |
| Ingenol mebutate | 0.5 | 0.5 | 0.5 | 0.5 | 0.5 |
| Benzyl Alcohol | 5 | 5 | 5 | 5 | 5 |
| Citrate buffer (1%) | 400 | | | | |
| pH 2.7 | | | | | |
| Citrate buffer (1%) | | 400 | 400 | 200 | 200 |
| pH 2.3 | | | | | |
| Sepineo P600 | 25 | 25 | 25 | 25 | 25 |
| Polysorbate 80 | 5 | 5 | 5 | 5 | 5 |
| Propylene glycol | 210 | 210 | 210 | 210 | 310 |
| Medium chain | 104.5 | 254.5 | 229.5 | 554.5 | 454.5 |
| triglycerides | | | | | |
| Isopropyl myristate | 200 | | | | |
| Macrogol 2 stearyl | 50 | | 50 | | |
| ether | | | | | |
| N-Methyl Pyrrolidine | | 50 | 50 | | |
| Menthol | | 50 | 25 | | |

Study protocol 15—Preparation method
1. Drug phase: PEP005 was solubilised in Benzyl Alcohol.
2. Buffer stock solution 1% (w/w): Citric acid and Na-Citrate were weighed and dissolved in the weighed amount of water. pH was adjusted with either Citric acid or NaOH solutions.
3. Aqueous phase: The Buffer stock solution, Propylene Glycol, IPA, Polysorbate 80 and Seppineo P600
4. Oil phase: Miglyol, Akoline, Imwitor, IPM, Macrogol 2 stearyl ether, NMP and Menthol were mixed (at 50° C.) until homogeneity. Then cooled down to 30° C.
5. Final formulation: The Drug phase was added to the Oil phase under mixing. Then the Aqueous phase was slowly added to the Oil phase. Homogenisation at room temperature.

### Examples 1E

### Study Protocol 45—Compositions

| | |
|---|---|
| 45-11A | 45-13A |

| | |
|---|---|
| Components | mg/g |

| | | |
|---|---|---|
| Ingenol | 0.5 | 0.5 |
| mebutate | | |
| Benzyl alcohol | 10 | 10 |
| Cetomacrogol | 890 | 590 |
| emulsifying | | |
| ointment (*) | | |
| Citrate buffer | 100 | 400 |

(*) Cetomacrogol emulsifying ointment is a mixture of 500 mg/g Paraffin white soft, 240 mg/g Cetostearyl alcohol, 60 mg/g cetomacrogol-100 and 200 mg/g Paraffin liquid.

Study protocol 45—Preparation method
1. Drug phase: PEP005 was solubilised in Benzyl Alcohol.
2. Buffer stock solution: Citric acid and Na-Citrate were weighed and dissolved in the weighed amount of water. pH was adjusted with either Citric acid or NaOH solutions.
3. Oil phase: Cetomacrogol emulsifying ointment was melted.
4. Final formulation: Buffer stock was slowly added to the oil phase under homogenisation with Silverson L4RT. Drug phase was then added to the o/w emulsion applying gentle stirring.

### Examples 1F

### Study Protocol 73—Compositions

Composition number: 130-296-73 (-14A, -15A, -16A)

| | Amount mg/g | | | |
|---|---|---|---|---|
| | o/w | o/w cream | o/w cream | |
| | cream | IPM | cyclomethicone | |
| Components | 14A | 15A | 16A | Function |
| PEP005 | 1 | 1 | 1 | API |
| Benzyl alcohol | 5 | 5 | 5 | Solvent |
| Sepineo P 600 | 25 | 25 | 25 | Gellant |
| PG | 210 | | | Emollient |
| MCT | 554.5 | | | Emollient |
| IPM | | 210 | | Emollient |
| Cyclomethicone | | | 554.5 | Emollient |
| Polysorbate 80 | 5 | 5 | 5 | Surfactant |
| Citrate buffer | 199.5 | 755.6 | 411.1 | Buffer |

### O/W Cream 1: Composition Number: 130-296-73 (-14A, -15A, -16A)

1. API stock: Weigh an appropriate amount of PEP005 into red cap flask containing the desired amount of benzyl alcohol. Stir until dissolution.
2. Buffer: weight out appropriate amount of Citric acid and Na-Citrate and water. Adjust pH to 2.9±0.2 with either Citric acid or NaOH of e.g. 0.5-1M. Store at 2-8° C.
3. Water phase: In a suitable container mix the buffer, Propylene Glycol (if excisted), polysorbate 80 and Sepineo P600 (Sepineo P600: remember to shake bottle well before weighing). Mix or Homogenize and let to swell. (In process control: clear solution).
4. Oil phase: In a suitable container weigh and mix with whisk: MCT, isopropyl myristate or cyclomethicone. Final formulation: Add API stock to the oil phase under mixing. Add then slowly the oil phase to the water phase under homogenisation (at low rate). When all oil phase is incorporated in the water phase, homogenise with Silverson for about 5 minute at 2000 to 5000 rpm at room temperature.
5. Storage: at 2-8° C.

### Examples 1G

### Study Protocol 76—Compositions

Amount mg/g

| Components | 76-01A | 76-02A | 76-03A | 76-04A |
|---|---|---|---|---|
| PEP005 | 0.5 | 0.5 | 0.5 | 0.5 |
| BA | 9 | 9 | 9 | 9 |
| Arlamol E | 100 | | 100 | |
| Lanol 1688 | | | | 100 |
| Cetostearyl Alcohol | 35 | | | |
| Steareth-2 | 30 | | | |
| Steareth-21 | 20 | | | |
| Sepineo SE 68 | | 150 | 50 | 50 |
| Glycerol 85% | 15 | 15 | 15 | 15 |
| Buffer pH 2.75 | ad 1000 | ad 1000 | ad 1000 | ad 1000 |

### Study Protocol 76—Preparation Method

### Buffer Stock

Weight out appropriate amount of Citric acid and Sodium citrate to make a stock solution, e.g. 0.5-1.0 L.
Set pH with NaOH of e.g. 0.5-1M.
Buffer stock solution should be stored at 2-8° C.

### API Stock Phase

Charge appropriate amount of API in small glass bottle with screw cap.
Add appropriate amount of Benzyl Alcohol.

### Final Formulation

Charge appropriate amount of oily solvent and surfactant components
Melt them together at 75° C.
Charge appropriate amount of aqueous buffer and glycerol and mix them
Heat to 75° C. the buffer solution
Add slowly the aqueous phase to the oil phase by homogenization with Silversson
Continue to homogenize the emulsion for 4 minutes at 4000 rpm
Let the emulsion cool down by manual mixing
Add the Benzylalcohol phase, when the temperature falls under 30° C.
Mix the cream by whisking until the benzylalcohol phase distributed homogeneously.

### Examples 1H

### Study Protocol 77—Compositions

| | Amount mg/g | | |
|---|---|---|---|
| Components | 77-01A | 77-02A | 77-03A |
| Ingenol mebutate | 0.5 | 0.5 | 0.5 |
| Benzyl alcohol | 9 | 9 | 9 |
| DC-Elastomer 10 | 40 | 40 | 40 |
| Dimethicone (Dow Corning | 60 | 60 | 60 |
| Q7-9120 Silicone fluid 20 cst) | | | |
| Sepigel 305 | 20 | 40 | 40 |
| Isopropyl myristate | 95 | 95 | 95 |
| Argan oil | | 50 | |
| Cetiol CC | | | 50 |
| Citrate buffer* | 775.5 | 725.5 | 725.5 |

| | Amount |
|---|---|
| | mg/g |
| Components | 77-09A |
| Ingenol mebutate | 0.5 |
| Benzyl alcohol | 9 |
| Glycerol 85% | 40 |
| Arlacel 170 | 50 |
| Xanthan gum | 10 |
| Isostearyl | 30 |
| isostearate | |
| Isopropyl myristate | 50 |
| MCT | 50 |
| Citrate buffer* | 760.5 |

Study protocol 77—production method

### 77-(01A, 02A, 03A)

1. Buffer stock: Prepare citrate buffer. When a homogenous solution is obtained, measure the pH and if necessary adjust to 2.9±10.2 using NaOH or citric acid solutions.
2. Oil phase: Mix (using a whisk) the oily components; DC-9040 silicone elastomer blend (or DC-Elastomer 10), Dimethicone 350, Sepigel 305 (shake container before use) and Isopropyl myristate (IPM). It is recommended to firstly weigh IPM and dimethicone since they are fluid, followed by Sepigel 305 and at last the DC-9040 (or DC-Elastomer 10).
3. Slowly add the homogenous oil phase in a constant thin beam to the buffer stock while mixing (with a whisk or paddle mixer, or homogenising with Silverson at very low rate e.g. 500 rpm). When all oil phase is incorporated, homogenise the emulsion using Silverson at approximately 1500 rpm for about 5 minutes.
4. API stock solution: Prepare a stock solution of ingenol mebutate and benzyl alcohol. Store the solution in a red cap flask protected from light and stored at 2-8° C.
5. Add the API stock solution and mix with a whisk for about 3 minutes.

### 77-09A

1. Buffer stock: Prepare citrate buffer. When a homogenous solution is obtained, measure the pH and if necessary adjust to 2.9±10.2 using NaOH or citric acid solutions.
2. Water phase: Disperse Xanthan gum in Glycerol 85%. Add the buffer phase slowly to the Xantahan-glycerol mixture while mixing with a whisk. Add then Arlacel LC (and/or Arlacel 170) and heat the mixture to around 80° C.
3. Oil phase: Weigh isostearyl isostearate, isopropyl myristate and MCT and mix with a whisk. Heat the mixture to around 80° C.
4. Slowly add the oil phase to the water phase (both having the same temperature±5° C.) while homogenising at a very low rate (e.g. 500-800 rpm) or using a paddle mixer. When all oil phase is incorporated, increase the homogenisation rate to around 1500 rpm for around 5 minutes.
5. Cool down to around 30° C.
6. API stock solution: Prepare a stock solution of ingenol mebutate and benzyl alcohol. Store the solution in a red cap flask protected from light and stored at 2-8° C.
7. Add the API stock solution and mix with a whisk for about 3 minutes.

### Examples 1I

### Study Protocol 78—Compositions

| | Amount mg/g |
|---|---|
| Components | 78-02A |
| Ingenol | 0.5 |
| mebutate | |
| Benzyl alcohol | 9 |
| Glycerol 85% | 30 |
| Versaflex V-150 | 80 |
| Isopropyl | 100 |
| myristate | |
| Lanol 1688 | 50 |
| Citrate buffer | 731 |

| | Amount mg/g | | |
|---|---|---|---|
| Components | 78-06A | 78-07A | 78-08A |
| Ingenol mebutate | 0.5 | 0.5 | 0.5 |
| Benzyl alcohol | 9 | 9 | 9 |
| Glycerol 85% | 30 | 30 | 30 |
| Brij S2 | 20 | | 10 |
| Brij S10 | | 25 | 40 |
| Brij S20 | 20 | | |
| Arlacel 983 | | 25 | |
| Cetostearyl alcohol | 25 | 30 | |
| Cetyl alcohol | 25 | | 25 |
| Isostearyl alcohol | 20 | | |
| Cetyl palmitate | 20 | 20 | 20 |
| MCT | | | 50 |
| Isopropyl myristate | 100 | 100 | |
| Citrate buffer | 730.5 | 760.5 | 815.5 |

### Study Protocol 78—Production Method

### 78-02A

1. Buffer stock: Prepare citrate buffer. When a homogenous solution is obtained, measure the pH and if necessary adjust to 2.9±0.2 using NaOH or citric acid solutions.
2. Water Phase: In an appropriate container, weigh out Versaflex V-150 and add Glycerol 85%. Mix the two components with a whisk until homogeneity. Add then slowly the weighed amount of the buffer stock. Mix well until dissolution (paddle mixer or whisk).
3. API stock solution: Prepare a stock solution of ingenol mebutate (0.5 mg/g) and Isopropylmyristate (100 mg/g).
4. Oil phase: Mix (using a whisk) the oily components; Isopropyl myristate stock solution and Lanol 1688.
5. Slowly add the homogenous oil phase in a constant thin beam to the water phase while mixing (with a whisk or paddle mixer, or homogenising with Silverson at very low rate e.g. 500 rpm). When all oil phase is incorporated, homogenise the emulsion using Silverson at approximately 1500 rpm for about 5 minutes.
6. Add benzyl alcohol and mix until homogeneity.

### 78-(06A, -07A, -08A)

1. Water Phase: Prepare citrate buffer. When a homogenous solution is obtained, measure the pH and if necessary adjust to 2.9±0.2 using NaOH or citric acid solutions. Add glycerol 85%
2. Oil phase: Weigh in an appropriate container the oil compatible components: emulsifiers, co-emulsifiers, “gelling agents”, oils: Brij (S20, S2, S10), Isopropyl myristate, cetyl alcohol, Cetostearyl alcohol, cetyl palmitate etc.
3. Heat both phases (separately) to 65-70° C.
4. Slowly add the homogenous oil phase in a constant thin beam to the water phase while mixing (with a whisk or paddle mixer, or homogenising with Silverson at very low rate e.g. 500 rpm). When all oil phase is incorporated, homogenise the emulsion using Silverson at approximately 1500 rpm for about 5 minutes. Note rate and duration of homogenisation in the production paper.
5. Stir gently (with a whisk) to cool (below 40° C.).
6. Active formulations: Add the API stock solution and mix with a whisk for about 3 minutes. API stock solution: Prepare a stock solution of ingenol mebutate and benzyl alcohol. Store the solution in a red cap flask protected from light and stored at 2-8° C.

### Examples 1J

### Study Protocol 81—Compositions

| | Amount mg/g | | |
|---|---|---|---|
| Components | 81-01A | 81-02A | 81-03A |
| PEP005 | 0.5 | 0.5 | 0.5 |
| BA | 9 | 9 | 9 |
| MCT | 50 | 100 | |
| Paraffin liquid | | | 130 |
| PEG 400 | 50 | 50 | |
| Lutrol F127 | 200 | 200 | |
| Polysorbate 60 | | | 30 |
| Sorbitane stearate | | | 20 |
| Cetostearylalcohol | | | 100 |
| Glycerol 85% | | | 15 |
| Buffer pH 2.75 | ad 1000 | ad 1000 | ad 1000 |

Amount mg/g

| Components | 81-05A | 81-06A |
|---|---|---|
| PEP005 | 0.5 | 0.5 |
| BA | 9 | 9 |
| Paraffin liquid | | 120 |
| Cremophor A6 | 15 | 15 |
| Cremophor A25 | 15 | 15 |
| Cetostearylalcohol | 70 | 70 |
| Glycerol 85% | 30 | 30 |
| Buffer pH 2.75 | ad 1000 | ad 1000 |

### Study Protocol 81—Production Method

### Buffer Stock

Weight out appropriate amount of Citric acid to make a stock solution, e.g. 0.5-1.0 L.
Set pH with NaOH of e.g. 0.5-1M.
Buffer stock solution should be stored at 2-8° C.

### API Stock Phase

Charge appropriate amount of API in small glass bottle with screw cap (preventing alcohol evaporation).
   Add appropriate amount of Benzyl Alcohol.

### Final Formulation

### Formulation 01, 02

Weigh appropriate amount of Lutrol F127, PEG 400 and buffer in a suitable baker
Mix them together at 5° C. until no solid particle can be seen
Let the composition adjust to room temperature
Slowly add MCT by mixing either manually or by homogenenizer.
Add the Benzylalcohol phase, when the temperature falls under 30° C.
Mix the cream by whisking until the benzylalcohol phase distributed homogeneously.

### Formulation 03

Charge appropriate amount of oily base and surfactant components (Par. Liq., Polysorbate, Span, cetostearyl alc.)
Melt them together at 75° C.
Charge appropriate amount of aqueous buffer and glycerol and mix them
Heat to 75° C. the buffer solution
Add slowly the aqueous phase to the oil phase by homogenization with Silversson
Continue to homogenize the emulsion for about 4 minutes at 4000 rpm
Let the emulsion cool down by manual mixing
Add the Benzyl alcohol phase, when the temperature falls under 30° C.
Mix the cream by whisking until the benzyl alcohol phase is distributed homogeneously.

### Formulation 05, 06

Charge appropriate amount of oil components and surfactant (Par. Liq., Cremophor, cetostearyl alc.)
Melt them together at 75° C.
Charge appropriate amount of aqueous buffer and glycerol and mix them
Heat to 75° C. the buffer solution
Add slowly the aqueous phase to the oil phase by homogenization with Silversson
Continue to homogenize the emulsion for about 4 minutes at 4000 rpm
Let the emulsion cool down by manual mixing
Add the Benzyl alcohol phase, when the temperature falls under 30° C.
Mix the cream by whisking until the benzyl alcohol phase distributed homogeneously.

### Example 2

### Results of Chemical Stability Studies

A number of compositions of the invention were tested for chemical stability. This testing required extraction of ingenol-2-angelate from the composition by dissolution in a solvent mixture of acetonitrile and phosphoric acid. Following extraction, organic impurities were identified using reversed phase UHPLC with UV detection at 220 nm. Following accelerated stability testing, the following compositions from Example 1B, 1C, 1D, and 1E were predicted to reach a storage period of 2 years at room temperature (25° C.), indicating that less than 10% of the ingenol-3-angelate would be expected to degrade over the 2 years and/or less than 5% by weight of total ingenanes in the composition would be expected to be ‘A’ and/or ‘B’.
1A: Compositions 68-01A, 68-17A, 68-19A
1B: Compositions 08-07A, 08-08A, 08-09A, 08-10A.
1C: Compositions 15-17A, 15-18A.
1D: Compositions 01A, 05A, 12A, 13A 17A, 18A, 21A, 22A, 23A, 24A.
76-01A, 76-02A, 76-03A
77-01A, 77-02A, 77-03A, 77-09A
78-06A, 78-07A, 78-08A
81-03A, 81-06A

### Example 3

To investigate the skin penetration and permeation of ingenol-3-angelate from compositions of the invention, a skin diffusion experiment was conducted. Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

Static Franz-type diffusion cells with an available diffusion area of 3.14 cm² and receptor volumes ranging from 8.6 to 11.1 ml were used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After one hour the saline was replaced by receptor medium, 0.04 M isotonic phosphate buffer, pH 7.4 (35° C.), containing 4% bovine serum albumin. Sink conditions were maintained at all times during the period of the study, i.e. the concentration of the active compounds in the receptor medium was below 10% of the solubility of the compounds in the medium.

The skin penetration experiment was allowed to proceed for 21 hours. Samples are then collected from the following compartments:

The stratum corneum was collected by tape stripping 10 times using D-Squame® tape (diameter 22 mm, CuDerm Corp., Dallas, Tex., USA). Each tape strip is applied to the test area using a standard pressure for 5 seconds and removed from the test area in one gentle, continuous move. For each repeated strop, the direction of tearing off was varied. The viable epidermis and dermis was then sampled from the skin in a similar fashion.

Samples (1 ml) of the receptor fluid remaining in the diffusion cell were collected and analysed.

The concentration of ingenol-3-angelate in the samples was determined by LC mass spectrometry.

### Results

FIG. 1 shows the results of the study.

### Example 4

### Local Skin Reaction (LSR) Model in Hairless Guinea Pigs in Compositions

In this model hairless guinea pigs are treated topically with the test compounds on dorsal skin on a field of 1-2 cm2 in size. Subsequently, erythema, oedema, wounding and vascular leakage (intracutaneous bleeding) is assessed visually and score on a 0 to 3 scale, where 0 means normal and 3 is the most severe reaction, typically extending outside the treatment area for eythema and oedema, or covering the entire treatment area for wounding and vascular leakage. The composite LSR score is calculated as the sum of the four individual parameters. Thus, the composite LSR has a range from 0 to 12. Typically, the composite LSR peaks 24 h-48 h after administration of ingenol derivatives. The composite LSR is used as a surrogate endpoint for efficacy, assuming that higher potency of the formulation will lead to higher efficacy. The composite LSR has been shown to correlate to the concentration of the API in the applied formulation (in-house data). Other surrogate endpoints in this model includes histological assessment of necrosis and inflammation in epidermis and dermis, and mRNA expression of CDSN, which in human trials have been shown to correlate inversely to the extend of epidermal necrosis (in-house data).

Results FIG. 2 shows the data from the study.

### Example 5

### Anti-Tumor Efficacy in B16 Melanoma Mice Model of Compositions

In this model B16 melanoma cells are injected intradermally in syngeneic C57BL/6 mice. After 3 days the mice are treated topically at the tumor bearing site once daily for two consecutive days with the test formulation. Tumor growth is monitored for 40 days and tumor size exceeding 250 mm3 or ulcerating tumor is used as termination point. Kaplan-Meyer survival curves is used to calculate efficacy of the test compound/formulation. This model is used as a surrogate for efficacy.

Results are shown in FIG. 3.

1. A topical composition for cutaneous application which is an oil-in-water emulsion comprising
(a) an oily phase,
(b) an ingenol derivative in dissolved form;
(c) an aqueous phase buffered to a pH of below 4.5,
(d) at least one surfactant.

2. The composition of claim 1, wherein the surfactant is selected from the following surfactant groups of PEG sorbitane fatty acid esters, PEG fatty alcohol ethers, PEG fatty acid esters, PEG castor oil derivatives, PEG glicerides, propylene glycol esters, sucrose esters, poloxamers and phospholipids.

3. The composition according to claim 1, wherein the surfactants are selected from the group polyethylene glycol C₆₋₂₀ fatty acid glyceride selected from the group consisting of caprylocaproyl PEG glyceride, lauroyl PEG glyceride, linoeoyl PEG glyceride, oleoyl PEG glyceride and stearoyl PEG glyceridepolyoxyethylene C₈₋₂₀ alkyl ether selected from the group consisting of PEG monocetyl ether, PEG monolauryl ether, PEG monooleyl ether and PEG monostearyl ether (such as polyoxyethylene-2-stearyl ether), a polysorbate selected from the group consisting of polysorbate 20, 40, 60 and 80, or a polyoxyethylene castor oil derivative such as polyoxyl castor oil or hydrogenated polyoxyl castor oil.

4. The composition according to claim 1, wherein the composition contains additional surfactants, wherein the additional surfactant is selected from fatty alcohol, sucrose esters, mono-di-tri-glyceride or polyglycerol derivatives.

5. The composition according to claim 4, wherein the surfactant is selected from cetostearyl alcohol, sucrose stearate and palmitate esters, caprylic/capric glycerides, glyceryl monooleate, and triglycerol diisostearate.

6. The composition of claim 1, wherein the surfactant is present in an amount of from about 0.1% by weight to about 20% by weight of the composition.

7. The composition according to claim 6, wherein the surfactant or surfactants are present in a total concentration of from about 0.5% by weight to about 8% by weight, or from about 1% by weight to about 7% by weight, such as about 2.5% by weight, of the composition.

8. The composition according to claim 1, wherein the ingenol derivative is selected from the group consisting of ingenol-3-angelate, ingenol-5-angelate, ingenol-20-angelate, 20-O-acetyl-ingenol-3-angelate and 20-deoxy-ingenol-3-angelate.

9. The composition according to claim 8, wherein the ingenol derivative is ingenol-3-angelate.

10. The composition according to claim 1, wherein the oily phase of the composition contains at least one solvent.

11. The composition according to claim 10, wherein the oily solvent is selected from vegetable oil, e.g. sesame oil, sunflower oil, palm kernel oil, corn oil, safflower oil, olive oil, avocado oil, jojoba oil, grape kernel oil, almond oil, canola oil, coconut oil, cottonseed oil, peanut oil, walnut oil, soybean oil or wheat germ oil, a highly purified vegetable oil, e.g. medium chain triglycerides, long chain triglycerides, castor oil, caprylic/capric mono- and diglycerides or caprylic/capric mono-, di- and triglycerides, a synthetic oil, e.g. isopropyl myristate, isopropyl palmitate, isopropyl linoleate, isopropyl monooleate, isostearyl isostearate or polyoxypropylene stearyl ether (such as polyoxypropylene-15-stearyl ether), a propylene glycol derivative such as propylene glycol dicaprylate dicaprate (such as Labrafac, Capryol 90 and Crodamol PC), and alkyl or dialkyl ester such as ethyl oleate, diisopropyl adipate, dicaprylyl carbonate or propylene carbonate, or a C₁₀₋₃₀ cholesterol or lanosterol ester, Silicone oils such as cyclomethicone 5-NF and dimethicone.

12. The composition according to claim 9, wherein the oily solvent is present in a concentration of about 0.5-80%, in particular 5-50%.

13. The composition according to claim 1 wherein the composition contains a co-solvent selected from the group consisting of alcohols such as benzyl alcohol, as ethanol, n-propanol, isopropanol, n-butanol, 2-butanol or benzyl alcohol, or diols such as propylene glycol, or fatty alcohols such as oleoyl alcohol, stearyl alcohol and cetyl alcohol.

14. The composition according to claim 1 further comprising a penetration enhancer such as a lower alcohols or diols, pyrrolidones, essential oils, azones, isopropyl esters, propylene glycol esters and surfactants.

15. The composition according to claim 14, wherein the penetration enhancer is selected from the group consisting of isopropylalcohol, isopropyl myristate, propylene glycol, menthol, n-methyl pyrrolidine (NMP) or squalene.

16. The composition according to claim 1 further comprising an occlusive agent selected from the group consisting of a mineral oil, e.g. liquid paraffin, or at least one paraffin selected from paraffins consisting of hydrocarbons with chain lengths from C₅ to C₆₀, the chain lengths peaking at C₁₄₋₁₆, C₁₈₋₂₂, C₂₀₋₂₂, C₂₀₋₂₆, C₂₈₋₄₀, and C₄₀₋₄₄, or mixtures thereof or an iso-paraffin such as isohexadecane or squalane, or petrolatum-polyethylene blend.

17. The composition according to claim 1, further comprising a viscosity-increasing ingredient.

18. A composition according to claim 17, wherein the viscosity-increasing ingredient is a polyacrylamide such as Sepineo P600, fumed silica such as aerosil, polysaccharides such as hyaluronic acid, chitosan, pectin, xanthan gum, agar, carrageenan, tragacanth, starch, polydextrose, cellulose derivative such as HEC, HPC, HPMC, MC, polyacrylates, polycarbophyl, polyvinylalcohol, polyvinylpirrolydones or PEG derivative such as PEG 100 distearate.

19. The composition according to claim 1, wherein the aqueous phase comprises 10-95% by weight, such as 20-90% by weight of the composition.

20. The composition according to claim 1, which further contains a stabiliser selected from NaCl, titanium dioxide, tocopheryl acetate, potassium sorbate or magnesium sulphate.

21. The composition according to claim 1 comprising about 0.001-0.5% by weight of the ingenol derivative.

22. The composition according to claim 1 for use in the treatment of a dermal disease or condition.

23. The composition of claim 22, wherein the dermal disease or condition is actinic keratosis, seborrheic keratosis, basal cell carcinoma, squamous cell carcinoma, warts, keloids, scars, photoaged or photodamaged skin, or acne.

24. The composition according to claim 1 wherein the composition is chemically stable.
